(19) United States
(12) Patent Application Publication
(10) Pub. No.: US 2026/0319110 A1
(43) Pub. Date: Oct. 15, 2026

(54) REAL-TIME MONITORING SYSTEM AND METHOD FOR RADON CONCENTRATION IN SMALL-FLOW AND DISCONTINUOUS UNDERGROUND WATER

(71) Applicant: WUHAN UNIVERSITY, Hubei (CN)

(72) Inventors: Jia-Qing Zhou, Hubei (CN); Zhi-Jie Che, Hubei (CN); Fu-Shuo Gan, Hubei (CN); Yi-Feng Chen, Hubei (CN)

(73) Assignee: WUHAN UNIVERSITY, Hubei (CN)

(21) Appl. No.: 19/464,670

(22) Filed: Jan. 30, 2026

(30) Foreign Application Priority Data
Feb. 7, 2025 (CN) 202510136228.1

(51) Int. Cl. G01T 1/167

(52) U.S. Cl. CPC G01T 1/167

(57) ABSTRACT

The present disclosure provides a real-time monitoring system and method for a radon concentration in small-flow and discontinuous underground water. The system includes a cabinet; a radon concentration measurement device; a flow-directing converging device, comprising a flow-directing tube, a water-converging chamber, and a water valve; and a collection-measurement integration device, comprising a central column, connected thereon with a third inlet gas pipe, a third outlet gas pipe and a third water drainage pipe, forming a horizontal angle of 90° between adjacent two of the pipes; a disk column; and a water-collecting bottle, where four water-collecting bottles are disposed on the disk column, and a first inlet water pipe, a first outlet gas pipe, a first inlet gas pipe, a fourth water drainage pipe and a gas exhaust mechanism are disposed on the water-collecting bottle. When the disk column rotates, the four water-collecting bottles are in different states.

### CROSS-REFERENCE TO RELATED APPLICATION

This application claims the priority benefit of China application no. 202510136228.1, filed on Feb. 7, 2025. The entirety of each of the above-mentioned patent applications is hereby incorporated by reference herein and made a part of this specification.

### TECHNICAL FIELD

The present disclosure relates to the technical field of nuclear radiation detection and in particular to a real-time monitoring system and method for a radon concentration in small-flow and discontinuous underground water.

### BACKGROUND OF RELATED ART

Radon (²²²Rn) is a radioisotope which widely found in the air, rock soil masses and water bodies in the environment. It is a colorless, odorless and water-soluble inert gas.

Geological disasters such as landslides, earthquakes and cave collapses caused by artificial or natural factors pose extreme threats to human life and property. In these geological disasters, water (for example, rainfall, periodic change of reservoir water level and excess pore water pressure of faults and the like) usually plays a critical induction effect; furthermore, the ground water is a main carrier for the transport of the rock and soil masses, and the physicochemical parameters of the ground water are important evolution indices of the rock and soil masses. Especially, the abnormal change of the radon concentration in the ground water usually indicates the evolution of the internal structure of the rock and soil masses and can be used as important warning index for the geological disasters.

In addition, along with further development of infrastructure construction and digging technologies in China, the tunnels and underground caves exponentially increase in scale and number. Further, because such underground structures have strong closeness and poor ventilation conditions, the problem of radon enrichment may occur during the digging and use stages. The high-concentration radon can severely harm personal health and therefore the radon concentration in the underground water of the tunnels becomes an important safety index. Whether during landslide, or earthquake or digging of the underground caves, the rock masses or soil masses may suffer deformation damage and even destruction. The occurring radon can be released into the surrounding underground water along with deformation damage of the rock and soil masses, leading to sharp increase of the radon concentration in the water flowing through the rock and soil masses. However, due to different permeabilities of different rock and soil masses and seasonal difference of atmospheric precipitation, the water sample flow may be spatiotemporally different. Thus, it is required to face the situation of small flow and discontinuity in the long-term monitoring of in-water radon.

On the one hand, for the water samples outcropping from some dense rock and soil masses, dripping usually occurs. In order to prevent radon gas from escaping due to water dripping, it is required to collect water samples close to the water outcropping points, which generally requires human labor to carry out. The collection and measurement work lasting up to several years can bring extremely high labor costs. Furthermore, the water monitoring points are usually located outdoors. Due to the temporal non-uniformity of the underground water flow, the human labor usually cannot ensure the timeliness of water sample collection and the real-timeness of measurement. Moreover, the long-term monitoring data collection density is relatively low, which is unfavorable for the long-term radon monitoring.

On the other hand, for the existing long-term monitoring methods for the water radon concentration, except for the above human collection monitoring, the continuous water is usually used as monitoring object and the water sample is released by pumping through nozzle to release radon from the water for measurement. This requires a given flow of the water sample and usually, a continuous flow is required to satisfy the real-time uninterrupted monitoring for radon in the underground water. In another method, the radon concentration monitoring is carried out in consideration of concentration balance of in-water radon and in-air radon rather than of the continuity of the water flow. But this monitoring method also requires a given flow, and the water in the device needs to be completely updated once in a period of time. Also, the response time for the change of the radon concentration is long. Hence, there may be a lag in responding to the sudden changes in radon concentration, which is detrimental to the prediction and early warning of earthquakes and landslides.

### SUMMARY OF THE INVENTION

In order to solve the problem of inefficient detection of radon concentration in small-flow underground water, the present application provides a real-time monitoring system and method for a radon concentration in small-flow and discontinuous underground water.

According to one aspect, the present application provides a real-time monitoring system for a radon concentration in small-flow and discontinuous underground water, which employs the following technical solution.

A real-time monitoring system for a radon concentration in small-flow and discontinuous underground water, comprising a cabinet, and a radon concentration measurement device, wherein the system further comprises:

a flow-directing converging device, comprising:

a flow-directing tube, comprises a trumpet-shaped opening, and a flow-directing pipe;

a water-converging chamber, disposed on the top of the cabinet to receive water dropping from the flow-directing tube; and

a water valve, comprises a movable ball valve and an outlet water pipe connected to the water-converging chamber;

a collection-measurement integration device, comprising:

a central column, connected thereon with a third inlet gas pipe, a third outlet gas pipe, and a third water drainage pipe, forming a horizontal angle of 90° between adjacent two of the third inlet gas pipe, the third outlet gas pipe, and the third water drainage pipe, wherein a bottom end of the third inlet gas pipe is connected to an output end of the radon concentration measurement device, and a bottom end of the third outlet gas pipe is connected to an input end of the radon concentration measurement device;

a disk column, rotatably disposed around the central column; and

a water-collecting bottle, wherein four water-collecting bottles are disposed and equidistantly distributed on the disk column, and a first inlet water pipe, a first outlet gas pipe, a first inlet gas pipe, a fourth water drainage pipe and a gas exhaust mechanism are disposed on the water-collecting bottle;

wherein when the disk column rotates, the four water-collecting bottles are in different states: only the first inlet water pipe of the water-collecting bottle in a water collection state is exactly below the outlet water pipe; the first outlet gas pipe of the water-collecting bottle in a measurement state is connected to the third outlet gas pipe, and the first inlet gas pipe is connected to the third inlet gas pipe; the fourth water drainage pipe of the water-collecting bottle in a water drainage state is connected to the third water drainage pipe.

Furthermore, the disk column is provided with four second inlet gas pipes, four second outlet gas pipes, and four second water drainage pipes; the four second inlet gas pipes are connected in one-to-one correspondence with the four first outlet gas pipes, the four second outlet gas pipes are connected in one-to-one correspondence with the four first inlet gas pipes, and the four second water drainage pipes are connected in one-to-one correspondence with the four fourth water drainage pipes;

a horizontal angle between adjacent two of the second inlet gas pipes, the second outlet gas pipes, and the second water drainage pipes is 90°; and

the second inlet gas pipes are at the same horizontal height as a top end of the third outlet gas pipe, the second outlet gas pipes are at the same horizontal height as a top end of the third inlet gas pipe, and the second water drainage pipes are at the same horizontal height as a top end of the third water drainage pipe.

Furthermore, a sealing waterproof layer is wrapped on a peripheral wall of the central column, a first hole connected to the top end of the third outlet gas pipe is disposed on the sealing waterproof layer, and the first hole has a hole diameter smaller than a pipe diameter of the second inlet gas pipes;

a second hole connected to the top end of the third inlet gas pipe is disposed on the sealing waterproof layer, and the second hole has a hole diameter smaller than a pipe diameter of the second outlet gas pipes; and

a third hole connected to the top end of the third water drainage pipe is disposed on the sealing waterproof layer, and the third hole has a hole diameter smaller than a pipe diameter of the second water drainage pipes.

Furthermore, a check valve is disposed on each of the first inlet water pipe, the first inlet gas pipe, the first outlet gas pipe and the fourth water drainage pipe; and

the check valve on the first inlet water pipe is configured to allow flow in a top-to-bottom direction, the check valve on outside of the first inlet gas pipe is configured to allow flow from outside of the bottle to inside of the bottle, the check valve on the first outlet gas pipe is configured to allow flow from inside of the bottle to outside of the bottle, and the check valve on the fourth water drainage pipe is configured to allow flow from inside of the bottle to outside of the bottle.

Furthermore, the gas exhaust mechanism comprises a gas-exhaust movable valve disposed on the top of the water-collecting bottle, at least two convex exhaust holes are disposed on the top of the gas-exhaust movable valve; a rubber column is slidably disposed inside the gas-exhaust movable valve; and a middle hole staggered with all of the several convex exhaust holes is disposed in the middle of the rubber column.

Furthermore, an end of the first inlet gas pipe penetrating into the water-collecting bottle is connected with a porous glass rod.

Furthermore, a second stepping motor and a water collection full-load signal processing device are disposed inside the cabinet, and an output end of the second stepping motor is in transmission connection with the disk column; and

the water collection full-load signal processing device comprises:

a graduated cylinder, wherein an extension opening is disposed at a side of the top of the graduated cylinder close to the disk column, and an overflowing opening is disposed at a side of the top of the first inlet water pipe away from the central column, and the extension opening is located below the overflowing opening on the water-collecting bottle in the water collection state;

a first pressure sensor, disposed on an inner wall of the graduated cylinder, and connected with the second stepping motor in a controlled way; and

a first water drainage pipe, connected to a sidewall of a bottom end of the graduated cylinder, wherein a maximum drainage flow of the first water drainage pipe is 0.045 ml/min.

Furthermore, the radon concentration measurement device comprises a columnar drying tube and a radon monitoring instrument, a gas inlet of the columnar drying tube is connected to the third outlet gas pipe, a gas outlet thereof is connected to a gas inlet of the radon monitoring instrument, and a gas outlet of the radon monitoring instrument is connected to the third inlet gas pipe.

Furthermore, a water drainage opening is connected at a side of the top of the water-converging chamber on the cabinet.

According to the second aspect, a real-time monitoring method for a radon concentration in small-flow and discontinuous underground water, which is based on the above real-time monitoring system for the radon concentration in the small-flow and discontinuous underground water, wherein the method comprises the following steps:

step S1, the trumpet-shaped opening of the flow-directing tube is fixed exactly below the outcropping water sample, and the lower end of the flow-directing tube is placed in the water-converging chamber;

step S2, the water sample flows through the water valve to the first inlet water pipe of the water-collecting bottle in the water collection state, and stored in the water-collecting bottle; at this time, none of the first outlet gas pipe, the first inlet gas pipe and the fourth water drainage pipe of the water-collecting bottle is forming a connection with the central column, and the gas in the water-collecting bottle is exhausted through the gas exhaust mechanism, and the water level of the water-collecting bottle increases continuously;

step S3, when the water level of the water-collecting bottle in the step S2 reaches a set value, the disk column is driven to rotate 90°; the water-collecting bottle enters the measurement state, and the first outlet gas pipe is connected to the third outlet gas pipe and the first inlet gas pipe is connected to the third inlet gas pipe; the radon escaping in the water-collecting bottle enters the radon concentration measurement device for radon concentration detection; the gas subjected to the detection flows through the third inlet gas pipe and the first inlet gas pipe and back to the water-collecting bottle;

step S4, after the radon concentration in the water-collecting bottle in the step S3 is detected, the disk column is driven to continue a rotation of 90° in the same direction; the water-collecting bottle enters the water drainage state, and the fourth water drainage pipe is connected to the third water drainage pipe; the water level in the bottle continuously decreases;

step S 5, the disk column is driven further to continue a rotation of 90° in the same direction for each step so that each water-collecting bottle on the disk column sequentially undergoes the steps S2 to S4, so as to realize the real-time radon concentration monitoring for the small-flow and discontinuous water sample.

In conclusion, the present disclosure includes at least one of the following beneficial technical effects.

1. In the present disclosure, by using multiple flow-directing tubes on the flow-directing converging device, convergence and collection for the small-flow and discontinuous water can be realized while the radon escape caused by dripping can also be reduced.

2. In the present disclosure, the disk column in the collection-measurement integration device sequentially rotates 90° to enable each water-collecting bottle to sequentially undergo the water collection state, measurement state, water drainage state and idle state and repeat them, so as to realize automatic water sample collection, monitoring and drainage, reducing the labor costs and effectively improving water sample collection and monitoring timeliness.

3. In the present disclosure, the radon concentration monitoring for the small-flow and discontinuous underground water can be realized. The flow required for general in-water continuous radon monitoring is 2-3L/min. The water flow in the present disclosure only needs to satisfy the fact that the water-collecting bottle is filled up before radon decay, namely, the flow is greater than 0.045 ml/min, significantly extending the flow range of the water sample measurement.

4. In the present disclosure, the automatic water collection and radon measurement with the collection-measurement integration device has its response time improved much as compared with the water-steam balanced discontinuous water sample radon measurement method. The general radon water-steam balance time is as shortest as about 2 hours, and one set of measurements can be completed within as shortest as about 30 min in the present disclosure.

5. In the present disclosure, by the control and data collection device, on the one hand, remote transmission and real-time analysis for the radon concentration data can be realized, and on the other hand, the system operation can be remotely controlled so that any device fault can be detected in time and monitoring can be stopped.

6. The present disclosure is a modular system. The removable power source can realize long-time outdoor monitoring for the radon concentration of the water sample while some of the batteries can be replaced without interrupting power supply, not affecting the normal operation of the system. Further, the removable and replaceable radon monitoring instrument is easy to repair and the system failure time can be reduced by replacement.

### BRIEF DESCRIPTION OF THE DRAWINGS

In order to more clearly describe the technical solutions in the present disclosure or in the prior arts, brief descriptions will be made below to the drawings required for descriptions of the embodiments or prior arts. Apparently, the drawings described below are only some embodiments of the present disclosure, and persons of ordinary skills in the arts can also obtain other drawings based on these drawings without carrying out creative work.

FIG. 1 is a schematic diagram illustrating an entire structure of an embodiment of the present disclosure.

FIG. 2 is a schematic diagram illustrating a front sectional structure of a collection-measurement integration device according to an embodiment of the present disclosure.

FIG. 3 is a schematic diagram illustrating a side sectional structure of a collection-measurement integration device according to an embodiment of the present disclosure.

FIG. 4 is a top view of the collection-measurement integration device in FIG. 3, a cross-sectional top view taken along the line I-I′, a cross-sectional top view taken along the line II-II′ and a cross-sectional top view taken along the line III-III′.

FIG. 5 is a structural schematic diagram illustrating a water-collecting bottle according to an embodiment of the present disclosure.

### NUMERALS OF DRAWINGS

1. flow-directing converging device, 11. flow-directing tube, 111. trumpet-shaped opening, 112. flow-directing pipe, 12. water-converging chamber, 121. water drainage opening, 122. downwardly-recessed basin, 13. water valve, 131. movable ball valve, 132. outlet water pipe, 14. steel caterpillar belt;
2. collection-measurement integration device;
21. water collection full-load signal processing device, 211. first water drainage pipe, 212. first pressure sensor, 213. graduated cylinder;
22. disk column, 221. second inlet gas pipe, 222. second outlet gas pipe, 223. second water drainage pipe;
23. central column, 231. sealing waterproof layer, 232. third inlet gas pipe, 233. third outlet gas pipe, 234. third water drainage pipe, 235. third hole, 236. first hole, and 237. second hole;
24. water-collecting bottle, 241. first inlet water pipe, 242. gas-exhaust movable valve, 2421. convex exhaust hole, 2422. rubber column, 243. first outlet gas pipe, 244. first inlet gas pipe, 2441. porous glass rod, 245. check valve, and 246. fourth water drainage pipe;
3. radon concentration measurement device, 31. columnar drying tube, 32. radon monitoring instrument;
4. control and data collection device, 41. wireless signal transceiver, 42. wireless data collector, 43. host, and 44. display;
5. first stepping motor, 6. power source, 7. second stepping motor, and 8. cabinet.

### DETAILED DESCRIPTION OF THE EMBODIMENTS

In order to make the objects, technical solutions and advantages of the present disclosure clearer, the technical solutions of the present disclosure will be fully and clearly described by referring to the drawings in the present disclosure. Apparently, the described embodiments are only some embodiments of the present disclosure rather than all embodiments. All other embodiments obtained by persons of ordinary skills in the arts without carrying out creative work shall fall within the scope of present disclosure.

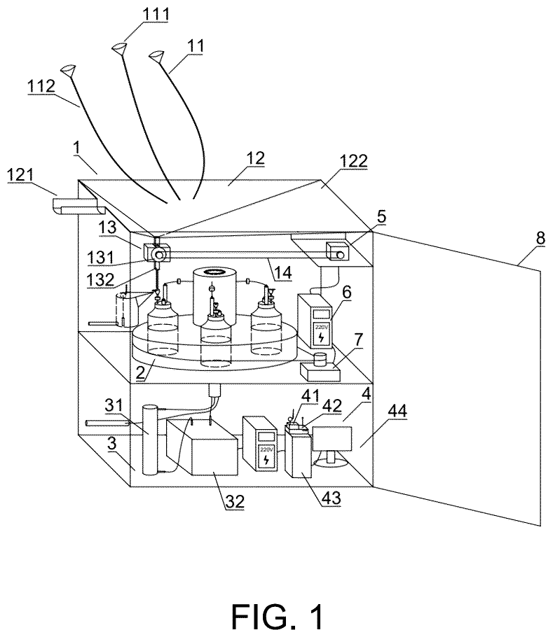

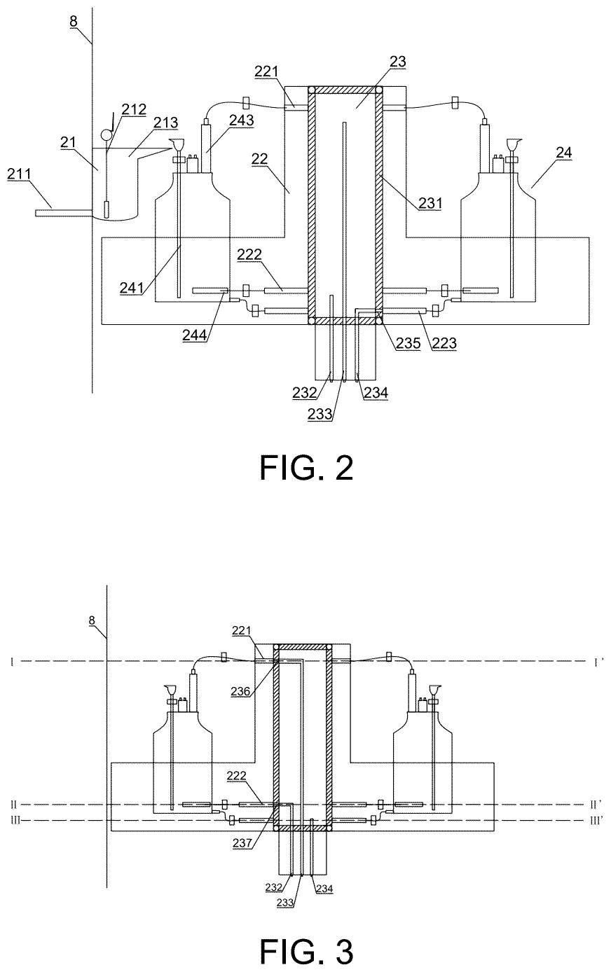

With reference to FIGS. 1 and 2, an embodiment of the present disclosure provides a real-time monitoring system for a radon concentration in small-flow and discontinuous underground water, which includes a cabinet 8, and a radon concentration measurement device 3. The cabinet 8 is wholly made of transparent sealing material, having good sealing and structural strength.

The system further includes:

a flow-directing converging device 1, which includes:

a flow-directing tube 11, including a trumpet-shaped opening 111 and a flow-directing tube body 112;

a water-converging chamber 12, disposed on the top of the cabinet 8 to receive water dropping from the flow-directing tube 11; where the water-converging chamber 12 includes a downwardly-recessed basin 122 disposed on the top of the cabinet 8 and a water drainage opening 121 connected at a side of the top of the downwardly-recessed basin 122; when a water flow is excessively large in the water-converging chamber 12, water is drained from the water drainage opening 121; and

a water valve 13, including a movable ball valve 131 and an outlet water pipe 132 connected to the water-converging chamber 12, where a first stepping motor 5 is disposed in the cabinet 8, and the movable ball valve 131 is connected by a stainless steel caterpillar belt to an output end of the first stepping motor 5, and is controlled to rotate by the first stepping motor 5, thereby turning on or off the water valve 13.

The system further includes a collection-measurement integration device 2, which, referring to FIGS. 2 and 3, includes:

a central column 23, connected thereon with a third inlet gas pipe 232, a third outlet gas pipe 233 and a third water drainage pipe 234, forming a horizontal angle of 90° between adjacent two of the third inlet gas pipe, the third outlet gas pipe, and the third water drainage pipe, where a bottom end of the third inlet gas pipe 232 is connected to an output end of the radon concentration measurement device 3, and a bottom end of the third outlet gas pipe 233 is connected to an input end of the radon concentration measurement device 3;

a disk column 22, rotatably disposed around the central column 23, where, specifically, the disk column 22 is separated from the central column 23, and upper and lower ends of the central column 23 and the disk column 22 are fixed by bearings; and

a water-collecting bottle 24, where four water-collecting bottles 24 are fixedly mounted and equidistantly distributed on the disk column 22, and a first inlet water pipe 241, a first outlet gas pipe 243, a first inlet gas pipe 244, a fourth water drainage pipe 246 and a gas exhaust mechanism are disposed on the water-collecting bottle 24.

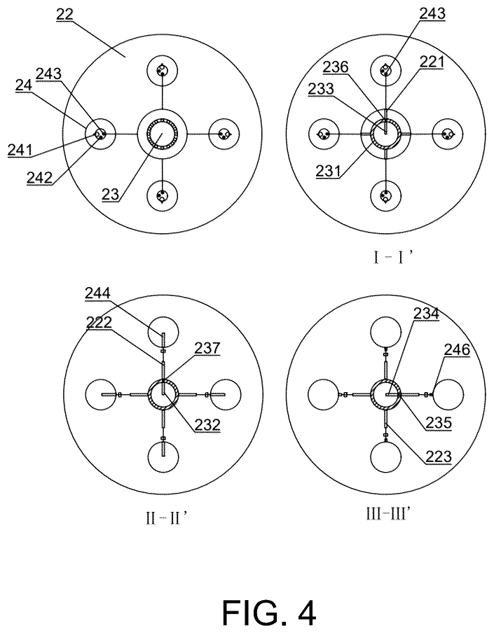

With reference to FIGS. 3 and 4, when the disk column 22 rotates, the four water-collecting bottles 24 are in different states: only the first inlet water pipe 241 of the water-collecting bottle 24 in a water collection state is exactly below the outlet water pipe 132; the first outlet gas pipe 243 of the water-collecting bottle 24 in a measurement state is connected to the third outlet gas pipe 233, and the first inlet gas pipe 244 is connected to the third inlet gas pipe 232; the fourth water drainage pipe 246 of the water-collecting bottle 24 in a water drainage state is connected to the third water drainage pipe 234; the water-collecting bottle 24 in an idle state is not connected to each of the outlet water pipe 132 and the central column 23.

The four water-collecting bottles 24 are all connected to the disk column 22 and relatively fixed positionally, which specifically means that: the disk column 22 is provided with four second inlet gas pipes 221, four second outlet gas pipes 222 and four second water drainage pipes 223; the four second inlet gas pipes 221 are connected in one-to-one correspondence with the four first outlet gas pipes 243, the four second outlet gas pipes 222 are connected in one-to-one correspondence with the four first inlet gas pipes 244, and the four second water drainage pipes 223 are connected in one-to-one correspondence with the four fourth water drainage pipes 246.

A horizontal angle between adjacent two of the second inlet gas pipes 221, the second outlet gas pipes 222 and the second water drainage pipes 223 is 90°, and the second inlet gas pipes 221 are at the same horizontal height as a top end of the third outlet gas pipe 233, the second outlet gas pipes 222 are at the same horizontal height as a top end of the third inlet gas pipe 232, and the second water drainage pipes 223 are at the same horizontal height as a top end of the third water drainage pipe 234.

Furthermore, a sealing waterproof layer 231 is wrapped on a peripheral wall of the central column 23 and the sealing waterproof layer 231 is fixed positionally relative to the central column 23. An end, close to the central column 23, of the second inlet gas pipes 221, the second outlet gas pipes 222 and the second water drainage pipes 223 on the disk column 22 is closely attached to an outer wall of the sealing waterproof layer 231, and slidable relative to the sealing waterproof layer 231. Further, the sealing waterproof layer 231 is made of elastic sealing material having good airtightness and strong wear resistance, such as butyl rubber or polyurethane rubber.

A first hole 236 connected to the top end of the third outlet gas pipe 233 is disposed on the sealing waterproof layer 231, and the first hole 236 has a hole diameter smaller than a pipe diameter of the second inlet gas pipes 221.

A second hole 237 connected to the top end of the third inlet gas pipe 232 is disposed on the sealing waterproof layer 231, and the second hole 237 has a hole diameter smaller than a pipe diameter of the second outlet gas pipes 222.

A third hole 235 connected to the top end of the third water drainage pipe 234 is disposed on the sealing waterproof layer 231, and the third hole 235 has a hole diameter smaller than a pipe diameter of the second water drainage pipes 223.

The first hole 236, the second hole 237 and the third hole 235 are respectively aligned in vertical height with the second inlet gas pipes 221, the second outlet gas pipes 222 and the second water drainage pipes 223, and are horizontally distributed in such a way that an interval of 90° is formed between adjacent holes. These holes are also respectively aligned with horizontal distribution positions of the four water-collecting bottles 24 on the disk column 22.

In addition, a check valve 245 is disposed on each of the first inlet water pipe 241, the first inlet gas pipe 244, the first outlet gas pipe 243 and the fourth water drainage pipe 246.

The check valve 245 on the first inlet water pipe 241 is configured to allow flow in a top-to-bottom direction, the check valve 245 on outside of the first inlet gas pipe 244 is configured to allow flow from outside of the bottle to inside of the bottle, the check valve 245 on the first outlet gas pipe 243 is configured to allow flow from inside of the bottle to outside of the bottle, and the check valve 245 on the fourth water drainage pipe 246 is configured to allow flow from inside of the bottle to outside of the bottle.

Furthermore, with reference to FIG. 1, the radon concentration measurement device 3 includes a columnar drying tube 31 and a radon monitoring instrument 32. The columnar drying tube 31 is made of acrylic transparent material, and a drying agent inside the columnar drying tube 31 is anhydrous cupric sulfate with indication function. A gas inlet of the columnar drying tube 31 is connected to the third outlet gas pipe 233, a gas outlet thereof is connected to a gas inlet of the radon monitoring instrument 32, and a gas outlet of the radon monitoring instrument 32 is connected to the third inlet gas pipe 232, so as to form a closed loop; the radon monitoring instrument 32 contains a miniature pump and controls turn-on or off and measurement of the miniature pump by receiving a wireless signal.

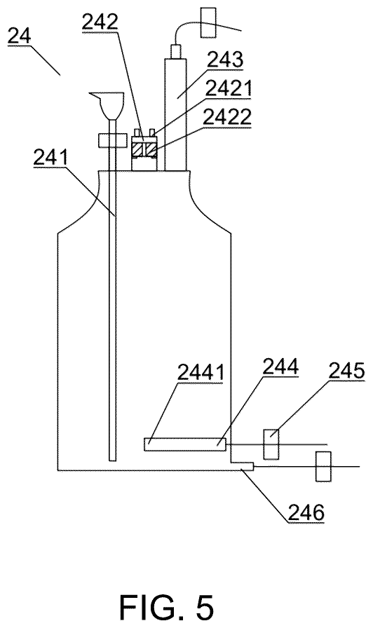

In addition, with reference to FIG. 5, the above gas exhaust mechanism includes a gas-exhaust movable valve disposed on the top of the water-collecting bottle 24; at least two convex exhaust holes 2421 are disposed on the top of the gas-exhaust movable valve 242; a rubber column 2422 is slidably disposed inside the gas-exhaust movable valve 242; and a middle hole staggered with all of the several convex exhaust holes 2421 is disposed in the middle of the rubber column 2422. The rubber column 2422 has a diameter slightly smaller than an inner diameter of a housing of the gas-exhaust movable valve 242. The middle hole of the rubber column 2422 has a diameter smaller than a net distance between two convex exhaust holes 2421. The rubber column 2422 has a density smaller than water density and thus can move up and down under water buoyancy in the gas-exhaust movable valve 242. Further, an end of the first inlet gas pipe 244 penetrating into the water-collecting bottle 24 is connected with a porous glass rod 2441 which is disposed to increase a contact between gas pumped by the radon monitoring instrument 32 and water in the water-collecting bottle 24.

Furthermore, in order to realize automatic measurement, with reference to FIGS. 1 and 2, a second stepping motor 7 and a water collection full-load signal processing device 21 are disposed inside the cabinet 8. An output end of the second stepping motor 7 is in transmission connection with the disk column 22, and specifically, the output end of the second stepping motor 7 is connected with the disk column 22 by a steel caterpillar belt 14 and the second stepping motor 7 can control a rotation angle of the disk column 22.

The water collection full-load signal processing device 21 includes:

a graduated cylinder 213, where an extension opening is disposed at a side of the top of the graduated cylinder 213 close to the disk column 22, and an overflowing opening is disposed at a side of the top of the first inlet water pipe 241 away from the central column 23, and the extension opening is located below the overflowing opening on the water-collecting bottle 24 in a water collection state;

a first pressure sensor 212, disposed on an inner wall of the graduated cylinder 213, and connected with the second stepping motor 7 and the first stepping motor 5 in a controlled way respectively; and

a first water drainage pipe 211, connected to a sidewall of a bottom end of the graduated cylinder 213, where a maximum drainage flow of the first water drainage pipe 211 is 0.045 ml/min; the first water drainage pipe 211 is disposed as a small-flow pipe to properly extend a time length in which the water-collecting bottle 24 in the water collection state remains in this state to ensure the time length is approximate to a time length of the water-collecting bottles 24 in the measurement state and water drainage state. Otherwise, when the water-collecting bottle in the water collection state is filled up and the water-collecting bottles in the measurement state and water drainage state are still in the process of measurement and drainage, the second stepping motor 7 controls the disk column 22 to rotate to perform state switching, affecting detection quality and subsequent process.

Specifically, with reference to FIG. 1, a control and data collection device 4 is disposed inside the cabinet 8. The control and data collection device 4 includes a wireless data collector, a wireless signal transceiver 41, a host 43, a display 44, and a power source 6. The power source 6 supplies power to the collection-measurement integration device 2, the radon concentration measurement device 3, and the control and data collection device 4 respectively. The power source 6 includes multiple batteries which are disposed as removable batteries so that a single battery can be replaced without power interruption.

Therefore, the central column 23 is connected to the disk column 22 in such a way that after the disk column 22 rotates, the water-collecting bottles at different positions are in different passageways, as shown in FIG. 4. Taking the arrangement orientation of the water-collecting bottles 24 in FIG. 2 as example, none of the first outlet gas pipe 243, the first inlet gas pipe 244 and the fourth water drainage pipe 246 of the water-collecting bottle 24 at the left side of the disk column 22 is connected to the central column 23. At this time, the water sample flows through the outlet water pipe 132 and then flows into the water-collecting bottle 24 through the first inlet water pipe 241, and the water-collecting bottle 24 is in a water sample collection state called water collection state. The first outlet gas pipe 243 and the first inlet gas pipe 244 of the water-collecting bottle 24 at the rear end of the disk column 22 (the left water-collecting bottle 24 shown in FIG. 3) are connected to the central column 23 through the first hole 236 and the second hole 237 respectively, and connected with the radon concentration measurement device 3; the first water drainage pipe 211 is not connected and the water-collecting bottle 24 is in a radon measurement state called measurement state; neither of the first outlet gas pipe 243 and the first inlet gas pipe 244 of the water-collecting bottle 24 at the right end of the disk column 22 is connected to the central column 23 but the first water drainage pipe 211 is connected to the third hole 235, so the water-collecting bottle 24 is in the water drainage state.

During a specific running process, when the first pressure sensor 212 sends a signal, the water-collecting bottle 24 in the water collection state completes water sample collection; when the radon concentration measurement device 3 sends radon concentration data, the water-collecting bottle 24 in the measurement state completes measurement, and the time is generally set to no more than 30 min; the water-collecting bottle 24 in the water drainage state controls the size of the third water drainage pipe 234, the third hole 235 and the second water drainage pipe 223 to set a minimum drainage flow, ensuring drainage is completed within 25 min. Such control involves setting the volume, height, and pipe diameter of the water-collecting bottle 24 to regulate the flow rate. For example, a 500 ml water collecting bottle with a 15 cm height that has to be emptied within 25 minutes, the minimum flow rate is 500 ml/1500 s=0.33 cm³/s. The flow rate Q relates to the bottle height and the drainage pipe cross-section through Q=μA\sqrt{2gh}, where μ is the flow coefficient (which can be conservatively set at 0.7), A is the cross-sectional area of the drainage pipe; g is the acceleration due to gravity, and h is the bottle height. For a conservative approach, Q can be set to 1 cm³/s (>0.33 cm³/s). This corresponds to a pipe diameter of approximately 1.5 mm, which can be set to 3 mm, to ensure complete drainage within 25 minutes. Only when the water-collecting bottle 24 in the water collection state and the water-collecting bottle 24 in the measurement state both complete respective operations can the control and data collection device 4 send a rotation signal to the second stepping motor 7 to drive the disk column 22 to rotate to switch the states of the water-collecting bottles 24. Meanwhile, when the disk column 22 rotates, the control and data collection device 4 can also send a rotation signal to the first stepping motor 5 so that the first stepping motor 5 drives the movable ball valve 131 to rotate so as to turn off the water valve 13; when the disk column 22 rotates to be in place, the control and data collection device 4 further sends a rotation signal to the first stepping motor 5 so that the first stepping motor 5 drives the movable ball valve 131 to rotate reversely so as to turn on the water valve 13.

Since holes are only opened at the specific positions of the sealing waterproof layer 231, when each of the water-collecting bottle 24 switches state, connection is only formed in the water-collecting bottle 24 arriving at the corresponding hole. During state switching process, the first outlet gas pipe 243, the first inlet gas pipe 244 and the fourth water drainage pipe 246 of the water-collecting bottle 24 are always sealed; the sealing waterproof layer 231 is closely filled between the disk column 22 and the central column 23 to ensure the sealing of the sealing waterproof layer 231 at the un-holed position.

Furthermore, the disk column 22 is rotated clockwise under the drive of the second stepping motor 7, and each of the water-collecting bottle 24 sequentially undergoes water sample collection, radon concentration measurement, water drainage and idling and repeats them. The third water drainage pipe 234, the third hole 235, the second water drainage pipe 223 and the pipes connecting them need to satisfy the minimum drainage flow of 25 ml/min.

An embodiment of the present disclosure further provides a real-time monitoring method for a radon concentration in small-flow and discontinuous underground water, which is based on the above real-time monitoring system for the radon concentration in the small-flow and discontinuous underground water. The method employs the following technical solution.

There is provided a real-time monitoring method for a radon concentration in small-flow and discontinuous underground water, which includes the following steps.

At step S1, the trumpet-shaped opening 111 of the flow-directing tube 11 is fixed exactly below the outcropping water sample, and the lower end of the flow-directing tube 11 is placed in the water-converging chamber 12; if there are several outcropping points in a same small area, several flow-directing tubes 11 are used to converge the water sample into the water-converging chamber 12.

At step S2, the water sample flows through the water valve 13 to the first inlet water pipe 241 of the water-collecting bottle 24 in the water collection state, and stored in the water-collecting bottle 24; at this time, none of the first outlet gas pipe 243, the first inlet gas pipe 244 and the fourth water drainage pipe 246 of the water-collecting bottle 24 is forming a connection with the central column 22, and the gas in the water-collecting bottle 24 is exhausted through the gas exhaust mechanism, and the water level of the water-collecting bottle increases continuously.

At step S3, when the water level of the water-collecting bottle 24 in the step S2 reaches a set value and is detected by the first pressure sensor 212, the second stepping motor 7 is controlled to drive the disk column 22 to rotate 90°. The water-collecting bottle 24 enters the measurement state, and the first outlet gas pipe 243 is connected to the third outlet gas pipe 233 and the first inlet gas pipe 244 is connected to the third inlet gas pipe 232. The radon escaping in the water-collecting bottle 24 enters the radon concentration measurement device 3 for radon concentration detection. The gas subjected to the detection is pumped by the miniature pump in the radon monitoring instrument 32 to flow through the third inlet gas pipe 232 and the first inlet gas pipe 244 and back to the water-collecting bottle 24 to ensure the gas pressure balance in the water-collecting bottle 24 and promote radon separation from water. Furthermore, During the radon concentration detection process, the wireless data collector records a date and a time of completing rotation by the second stepping motor 7, and the wireless signal transceiver 41 sends a pump operation signal to the radon monitoring instrument 32 and the radon monitoring instrument 32 pumps gas into the water-collecting bottle 24 to carry the radon in the water of the water-collecting bottle 24 into the radon monitoring instrument 32, start radon concentration measurement, and record a measurement result. The monitoring for the in-water radon concentration needs 30 min.

At step S4, after the radon concentration in the water-collecting bottle 24 in the step S3 is detected, the disk column 22 is driven to continue a rotation of 90° in the same direction. The water-collecting bottle 24 enters the water drainage state, and the fourth water drainage pipe 246 is connected to the third water drainage pipe 234. The water level in the bottle continuously decreases.

At step S5, the disk column 22 is driven further to continue a rotation of 90° in the same direction for each step so that each water-collecting bottle 24 on the disk column 22 sequentially undergoes the steps S2 to S4 and repeats them, so as to realize the real-time radon concentration detection for the small-flow and discontinuous water sample. The start mechanism for each rotation of 90° of the disk column 22 is as follows: when each water-collecting bottle 24 in the water collection state is filled up with water and the water overflows into the graduated cylinder 213, which is controlled by the water collection full-load signal processing device 21, and the radon concentration measurement device 3 is required to send a measurement completion signal, and thus a synchronous control is performed by both of them. Further, when the disk column 22 rotates, the first stepping motor 5 is also controlled to drive the movable ball valve 131 to rotate to turn off the water valve 13, and after the disk column 22 rotates to be in place, the first stepping motor 5 is controlled again to drive the movable ball valve 131 to rotate reversely to turn on the water valve 13.

Finally, it should be noted that the above embodiments are used only to describe the technical solutions of the present disclosure rather than to limit the present disclosure. Although detailed descriptions are made to the present disclosure by referring to the preceding embodiments, persons of ordinary skills in the arts should understand that they can still make modifications to the technical solutions recorded in each of the above embodiments or make equivalent replacement for partial technical features therein; these modifications or replacements do not cause the essence of the corresponding technical solutions to depart from the spirit and scope of the technical solutions of each embodiment of the present disclosure.

What is claimed is:

1. A real-time monitoring system for a radon concentration in small-flow and discontinuous underground water, comprising a cabinet, and a radon concentration measurement device, wherein the system further comprises:
a flow-directing converging device, comprising:
a flow-directing tube, comprises a trumpet-shaped opening and a flow-directing pipe;
a water-converging chamber, disposed on the top of the cabinet to receive water dropping from the flow-directing tube; and
a water valve, comprises a movable ball valve and an outlet water pipe connected to the water-converging chamber;
a collection-measurement integration device, comprising:
a central column, connected thereon with a third inlet gas pipe, a third outlet gas pipe, and a third water drainage pipe, forming a horizontal angle of 90° between adjacent two of the third inlet gas pipe, the third outlet gas pipe, and the third water drainage pipe, wherein a bottom end of the third inlet gas pipe is connected with an output end of the radon concentration measurement device, and a bottom end of the third outlet gas pipe is connected to an input end of the radon concentration measurement device;
a disk column, rotatably disposed around the central column; and
a water-collecting bottle, wherein four water-collecting bottles are disposed and equidistantly distributed on the disk column, and a first inlet water pipe, a first outlet gas pipe, a first inlet gas pipe, a fourth water drainage pipe and a gas exhaust mechanism are disposed on the water-collecting bottle;
wherein when the disk column rotates, the four water-collecting bottles are in different states: only the first inlet water pipe of the water-collecting bottle in a water collection state is exactly below the outlet water pipe; the first outlet gas pipe of the water-collecting bottle in a measurement state is connected to the third outlet gas pipe, and the first inlet gas pipe is connected to the third inlet gas pipe; the fourth water drainage pipe of the water-collecting bottle in a water drainage state is connected to the third water drainage pipe.

2. The real-time monitoring system for the radon concentration in the small-flow and discontinuous underground water according to claim 1, wherein the disk column is provided with four second inlet gas pipes, four second outlet gas pipes and four second water drainage pipes; the four second inlet gas pipes are connected in one-to-one correspondence with the four first outlet gas pipes, the four second outlet gas pipes are connected in one-to-one correspondence with the four first inlet gas pipes, and the four second water drainage pipes are connected in one-to-one correspondence with the four fourth water drainage pipes;
a horizontal angle between adjacent two of the second inlet gas pipes, the second outlet gas pipes and the second water drainage pipes is 90°;
the second inlet gas pipes are at the same horizontal height as a top end of the third outlet gas pipe, the second outlet gas pipes are at the same horizontal height as a top end of the third inlet gas pipe, and the second water drainage pipes are at the same horizontal height as a top end of the third water drainage pipe.

3. The real-time monitoring system for the radon concentration in the small-flow and discontinuous underground water according to claim 2, wherein a sealing waterproof layer is wrapped on a peripheral wall of the central column, a first hole connected to the top end of the third outlet gas pipe is disposed on the sealing waterproof layer, and the first hole has a hole diameter smaller than a pipe diameter of the second inlet gas pipes;
a second hole connected to the top end of the third inlet gas pipe is disposed on the sealing waterproof layer, and the second hole has a hole diameter smaller than a pipe diameter of the second outlet gas pipes; and
a third hole connected to the top end of the third water drainage pipe is disposed on the sealing waterproof layer, and the third hole has a hole diameter smaller than a pipe diameter of the second water drainage pipes.

4. The real-time monitoring system for the radon concentration in the small-flow and discontinuous underground water according to claim 1, wherein a check valve is disposed on each of the first inlet water pipe, the first inlet gas pipe, the first outlet gas pipe and the fourth water drainage pipe; and
the check valve on the first inlet water pipe is configured to allow flow in a top-to-bottom direction, the check valve on outside of the first inlet gas pipe is configured to allow flow from outside of the bottle to inside of the bottle, the check valve on the first outlet gas pipe is configured to allow flow from inside of the bottle to outside of the bottle, and the check valve on the fourth water drainage pipe is configured to allow flow from inside of the bottle to outside of the bottle.

5. The real-time monitoring system for the radon concentration in the small-flow and discontinuous underground water according to claim 1, wherein the gas exhaust mechanism comprises a gas-exhaust movable valve disposed on the top of the water-collecting bottle, at least two convex exhaust holes are disposed on the top of the gas-exhaust movable valve; a rubber column is slidably disposed inside the gas-exhaust movable valve; and a middle hole staggered with all of the several convex exhaust holes is disposed in the middle of the rubber column.

6. The real-time monitoring system for the radon concentration in the small-flow and discontinuous underground water according to claim 1, wherein an end of the first inlet gas pipe penetrating into the water-collecting bottle is connected with a porous glass rod.

7. The real-time monitoring system for the radon concentration in the small-flow and discontinuous underground water according to claim 1, wherein a second stepping motor and a water collection full-load signal processing device are disposed inside the cabinet, and an output end of the second stepping motor is in transmission connection with the disk column; and
the water collection full-load signal processing device comprises:
a graduated cylinder, wherein an extension opening is disposed at a side of the top of the graduated cylinder close to the disk column, and an overflowing opening is disposed at a side of the top of the first inlet water pipe away from the central column, and the extension opening is located below the overflowing opening on the water-collecting bottle in the water collection state;
a first pressure sensor, disposed on an inner wall of the graduated cylinder, and connected with the second stepping motor in a controlled way; and
a first water drainage pipe, connected to a sidewall of a bottom end of the graduated cylinder, wherein a maximum drainage flow of the first water drainage pipe is 0.045 ml/min.

8. The real-time monitoring system for the radon concentration in the small-flow and discontinuous underground water according to claim 1, wherein the radon concentration measurement device comprises a columnar drying tube and a radon monitoring instrument, a gas inlet of the columnar drying tube is in communication with the third outlet gas pipe, a gas outlet thereof is in communication with a gas inlet of the radon monitoring instrument, and a gas outlet of the radon monitoring instrument is connected to the third inlet gas pipe.

9. The real-time monitoring system for the radon concentration in the small-flow and discontinuous underground water according to claim 1, wherein a water drainage opening is connected at a side of the top of the water-converging chamber on the cabinet.

10. A real-time monitoring method for a radon concentration in small-flow and discontinuous underground water, which is based on the real-time monitoring system for the radon concentration in the small-flow and discontinuous underground water according to claim 1, wherein the method comprises the following steps:
step S1, the trumpet-shaped opening of the flow-directing tube is fixed exactly below the outcropping water sample, and a lower end of the flow-directing tube is placed in the water-converging chamber;
step S2, the water sample flows through the water valve to the first inlet water pipe of the water-collecting bottle in the water collection state, and stored in the water-collecting bottle; at this time, none of the first outlet gas pipe, the first inlet gas pipe and the fourth water drainage pipe of the water-collecting bottle is forming a connection with the central column, and the gas in the water-collecting bottle is exhausted through the gas exhaust mechanism, and the water level of the water-collecting bottle increases continuously;
step S3, when the water level of the water-collecting bottle in the step S2 reaches a set value, the disk column is driven to rotate 90°; the water-collecting bottle enters the measurement state, and the first outlet gas pipe is connected to with the third outlet gas pipe and the first inlet gas pipe is connected to the third inlet gas pipe; the radon escaping in the water-collecting bottle enters the radon concentration measurement device for radon concentration detection; the gas subjected to the detection flows through the third inlet gas pipe and the first inlet gas pipe and back to the water-collecting bottle;
step S4, after the radon concentration in the water-collecting bottle in the step S3 is detected, the disk column is driven to continue a rotation of 90° in the same direction; the water-collecting bottle enters the water drainage state, and the fourth water drainage pipe is connected to the third water drainage pipe; the water level in the bottle continuously decreases;
step S5, the disk column is driven further to continue a rotation of 90° in the same direction for each step so that each water-collecting bottle on the disk column sequentially undergoes the steps S2 to S4, so as to realize the real-time radon concentration monitoring for the small-flow and discontinuous water sample.
